(19) United States
(12) Patent Application Publication
(10) Pub. No.: US 2002/0174872 A1
(43) Pub. Date: Nov. 28, 2002

(54) Device for blocking a patient's view of a needle or a catheter

(76) Inventors: David R. Cyphers, Talent, OR (US); Candace A. Ruitenberg, Midland Park, NJ (US)

Correspondence Address: OSTROLENK FABER GERB & SOFFEN, 1180 AVENUE OF THE AMERICAS, NEW YORK, NY, 100368403

(21) Appl. No.: 10/127,128

(22) Filed: Apr. 19, 2002

Related U.S. Application Data
(60) Provisional application No. 60/286,059, filed on Apr. 23, 2001.

(51) Int. Cl. A61F 13/00

(52) U.S. Cl. 128/888

(57) ABSTRACT

A device that blocks a patient's view of the site of insertion of a needle or catheter while allowing a healthcare worker visual and physical access to the site. The devices includes a strap and an opaque member connected to the strap. The opaque member is made from a semi-rigid material. A portion of the opaque member may be lifted to provide a visual barrier that blocks the patient's view of the site of insertion of a needle or catheter.

### RELATED APPLICATIONS

This application claims the benefit of U.S. Provisional Application No. 60/286,059, which was filed on Apr. 23, 2001, the disclosure of which is incorporated herein by this reference.

### BACKGROUND OF THE INVENTION

The present invention relates to medical devices, and more specifically to devices that prevent a patient from viewing the site of insertion of a needle or catheter.

In hospitals or attended home care environments, the introduction of fluids or withdrawal of the same from a patient's body may be required as part of the treatment. This may require the use of catheters and other instruments such as syringes which penetrate the body of the patient. Viewing the site of insertion of a catheters or the needle of a syringe may frighten some patients thereby causing anxiety. To reduce the anxiety caused by viewing the site of insertion of the needle of a syringe or a catheter various devices have been proposed. The primary function of such devices is to block the patient's view of the site of insertion of the needle or catheter in order to reduce the patient's anxiety.

U.S. Pat. No. 5,131,412 proposes a device that provides a shield for a toddler's or an infant's arm, hand, leg or foot during an intravenous injection. This device consists of a mold that is covered with a disposable sleeve-like covering to provide a soft, non-allergic surface to protect the sensitive skin of the patient. This device is connected to the child as a long, tubular sleeve that is connected and held in place by an engaging means along each edge. An object of this device is to reduce a pediatric patient's anxiety caused by viewing the site of insertion of a catheter. A drawback of the device proposed by U.S. Pat. No. 5,131,412 is that, once in use, it obstructs visual and physical access to the site of insertion.

U.S. Pat. No. 5,634,906 proposes a device that effectively hides the needle during an injection. A hiding shield is placed on a metering syringe to minimize the anxiety that is experienced by the patient when viewing the site of the injection. This needle hiding shield comprises an opaque outer sleeve guard that hides the needle from the patient.

U.S. Pat. No. 6,283,126 proposes a device which is intended to be worn by a patient to prevent the unintended removal of needles or catheters from the patient, scratching of wounds, or removal of bandages. The device proposed by U.S. Pat. No. 6,283,126 includes a wrist band that secures the device on the wrist of a patient and a frustoconical member. The primary function of the frustoconical member is to prevent the patient from using his fingers to grasp and pull out any nasal, gastric, intravenous and other tubes. For this purpose, the frustoconical member extends beyond the fingers of the patient and is conically shaped to obstruct any physical interference from all sides. This aspect of the device shown in U.S. Pat. No. 6,283,126 makes it difficult for a healthcare worker to reach, view or adjust a catheter in that the physical path to the area of insertion is restricted by the frustoconical shape. In addition, U.S. Pat. No. 6,283,126 suggests that the frustoconical member may be made out of transparent material thus failing to recognize that the anxiety of a patient may be reduced by blocking the site of insertion of a needle or a catheter.

### SUMMARY OF THE INVENTION

An object of the present invention is to block a patient's view of the site of insertion of a needle or catheter in order to reduce the patient's anxiety.

Another object of the present invention is to block the patient's view of the site of insertion of a needle or catheter while allowing visual and physical access to the site.

According to the present invention, a device is provided which blocks a patient's view of the site of insertion of a catheter or needle. The device according to the present invention includes a strap which can be easily secured to and removed from a patient's limb.

According to an aspect of the invention an opaque member, which is attached to the strap, acts as a visual shield to block the view of the patient of the site of insertion of a needle or catheter.

According to another aspect of the invention the opaque member is made from a semi-rigid material so that it may be bent along a flexible portion of its body. The flexible portion constitutes a hinge that allows the opaque member to be lifted from a position where it is disposed about the curvature of the patient's arm to a substantially upright position where it may block the patient's view of the site of insertion of a needle or catheter.

According to another aspect of the invention a fanciful image may be disposed on a surface of the opaque member in the field of vision of the patient. The image so disposed may distract and/or amuse the patient thereby further reducing the patient's anxiety. This aspect of the invention may be particularly beneficial when the patient is a child or an infant.

According to another aspect of the invention, the opaque member may be made from a semi-rigid, opaque cloth sewn at an edge thereof to the strap.

According to another aspect of the invention the strap and the opaque member may be made from the same semi-rigid, opaque material.

According to yet another aspect of the invention, the semi-rigid, opaque material used in the device may be disposable and inexpensive, thereby making the device inexpensive.

A device according to the present invention includes few parts which are easily assembled together; thus the cost of manufacturing the device is low.

Other features and advantages of the present invention will become apparent from the following description of the invention which refers to the accompanying drawings.

### BRIEF DESCRIPTION OF THE DRAWINGS

FIG. 1 shows a view of a device for blocking a patient's view according to an embodiment of the present invention.

FIG. 2 shows the device of FIG. 1 secured on a patient's limb.

FIG. 3 shows the device of FIG. 1 as used to block the patient's view of the site of insertion of the needle of a syringe.

FIG. 4 shows the device of FIG. 1 as used to block the patient's view of the site of insertion of a catheter.

FIG. 5 shows the device of FIG. 1 as used to block an infant's view of the site of insertion of a catheter.

### DETAILED DESCRIPTION OF THE DRAWINGS

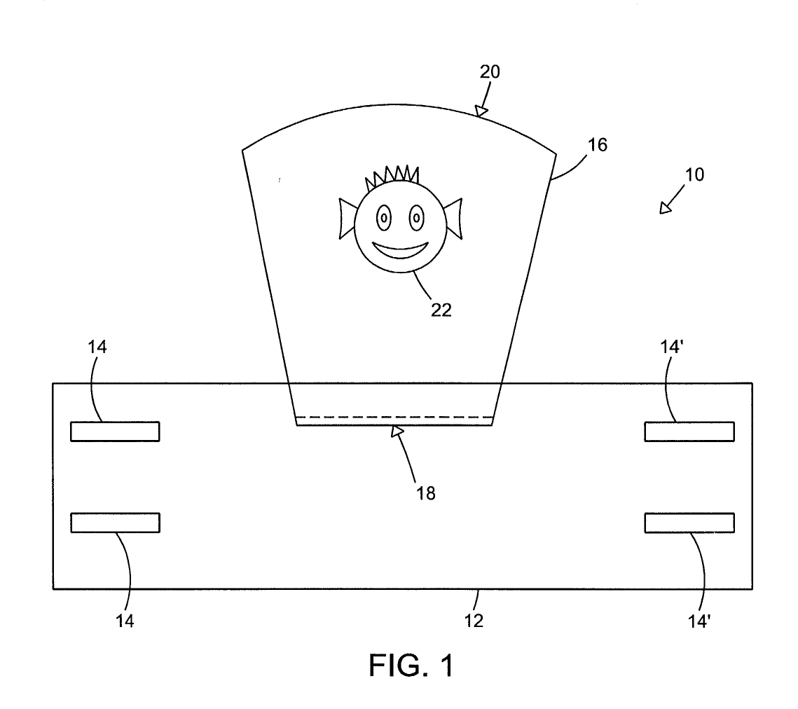

FIG. 1 shows a top view of an embodiment of a device 10 for preventing a patient from viewing the site of insertion of the needle of a syringe or a catheter according to the present invention. Device 10 includes strap 12 which serves as a means for securing apparatus 10 to a limb of a patient. Strap 12 includes fasteners 14, 14′ disposed at either end thereof. Fasteners 14, 14′ are preferably of the variety that are coupled to one another to secure strap 12 around a limb of a patient. Suitable fasteners for this purpose may be adhesive type fasteners comprising an adhesive surface such as adhesive tape, or snap connectors comprising of a male portion that mates with a respective female portion. In the preferred embodiment, a fastener sold under the trademark Velcro® is used. This type of fastener is desirable because it can be readily fastened and unfastened thereby allowing for easy adjustment. Device 10 also includes opaque member 16. Opaque member 16 is attached along edge 18 thereof to strap 12. In the preferred embodiment, opaque member 16 is shaped like a trapezium having an arcuate side 20 opposite to edge 18. Other shapes, however, can also be used as long as it is capable of blocking a patient's view of the site of insertion of a needle or catheter as described later.

Opaque member 16 is made from a semi-rigid material so that it is soft enough to be bent with little effort. A preferred material for this purpose is fabric interfacing. Fabric interfacing is a material often used in the construction of respirators. Fabric interfacing is a preferred material because it is opaque, it is semi-rigid, and it is cloth-like so that it may be sewn to strap 12. Also, fabric interfacing is relatively inexpensive so it can be used to make both strap 12 and opaque member 16, thereby rendering device 10 disposable. Strap 12 is made to a sufficient length to wrap around a limb of a patient, such as an arm of a patient. In a preferred embodiment, strap 12 is made long enough to permit an area of overlap at the ends thereof. Preferably, fasteners 14, 14′ are disposed in this area of overlap.

In one embodiment of the present invention, strap 12 is comprised of a material double-layered and sewn to one another according to a desired pattern. In another embodiment of the present invention the affixing strap may be made of a single layer of material. Opaque member 16 is preferably sewn to one side of strap 12 approximately at a central position.

A fanciful design 22 may be disposed on a surface of opaque member 16. The purpose of fanciful design 22 is to amuse the patient during the medical procedure; (for example, during penetration of the needle of a syringe). Providing fanciful design 22 is particularly useful when the patient is young.

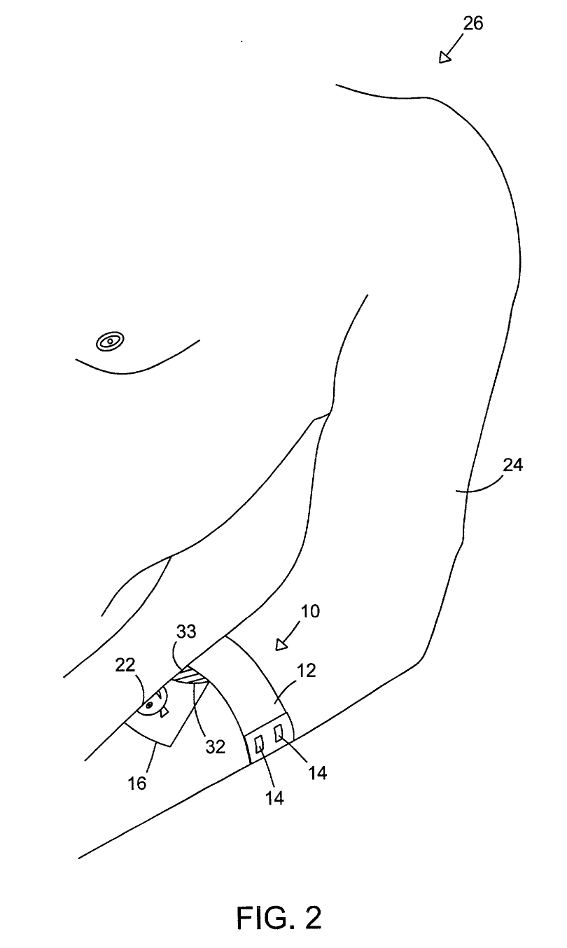

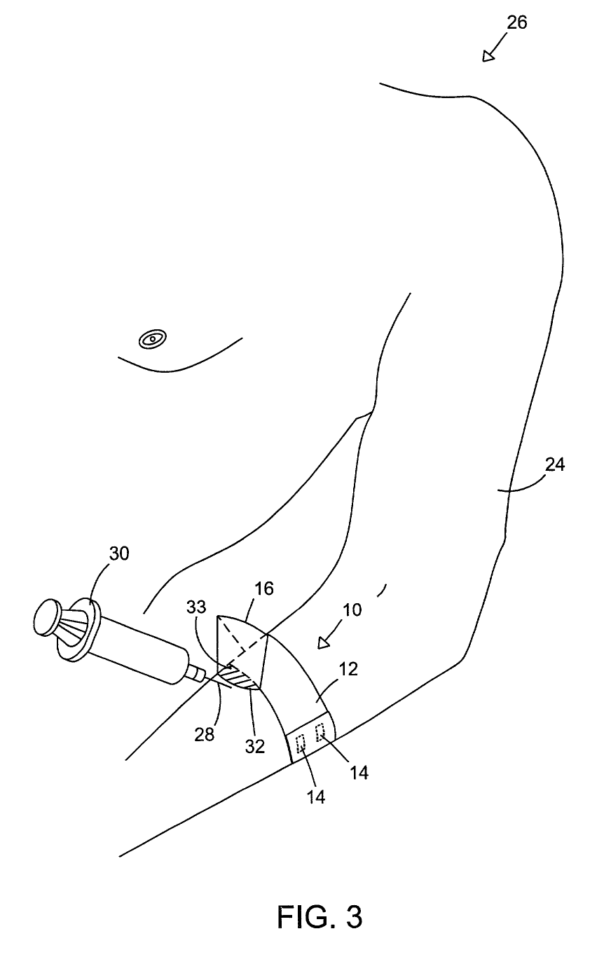

Referring now to FIGS. 2 and 3, device 10 is used in the following manner to prevent the patient from viewing the site of insertion of the needle of a syringe or a catheter. Referring first to FIG. 2, strap 10 is secured around limb 24 of patient 26 between the field of vision of patient 26 and the site of insertion of a needle or catheter. Device 10 is preferred for use on an arm of patient 26, but may also be used on a leg where appropriate. Once strap 12 is secured around limb 24 of patient 26, opaque member 16 lies on and may substantially conform to the curvature around limb 24.

Referring now to FIG. 3, a portion of opaque member 16 is lifted up to a substantially upright position to block the view of an area on limb 24 of patient 26 which is to be penetrated, for example, by needle 28 of a syringe 30. To be substantially upright does not necessarily mean that portion of opaque member 16 that is lifted must be normal to limb 24 of patient 26; rather, it means that a portion of opaque member 16 is lifted to a position where it may block the view of patient 26 of the site of insertion of, for example, needle 28 of syringe 30.

When opaque member 16 is lifted it is preferably bent along flexible portion 32. In its lifted position, opaque member 16 will have a base portion 33 (shaded region) having flexible portion 32 as one boundary thereof which terminates at strap 12. Base portion 33 lies on and substantially conforms to the curvature of limb 24 while the remaining portion of opaque member 16 is substantially upright relative to limb 24 to block the view of patient 26. Once lifted, the substantially upright portion of opaque member 16 will form a visual barrier that blocks the view of patient 26 of the site of penetration of a needle or a catheter. This visual barrier may have a concave or a convex curved surface due to the fact that flexible portion 32 of opaque member 16 is curved.

It should be noted that the semi-rigid characteristic of the material used for forming opaque member 16 allows for flexible portion 32 to be used as a hinge while preventing opaque member 16 from collapsing once it has been lifted to a substantially upright position.

It should also be noted that when a portion of opaque member 16 is lifted patient 26 is prevented from viewing the site of penetration while a healthcare worker can have access to the site of penetration for the purposes of observation and adjustment of the associated apparatus, e.g., catheter. Advantageously, for example, unlike other devices that are used to block the patient's view, the area that is penetrated is not covered by opaque member 16; therefore, the color of the skin of patient 26 may be observed when device 10 is in use. Thus, device 10 blocks the patient's view of the site of insertion of a needle or catheter while concurrently allowing the healthcare provider visual and physical access to the insertion site and the associated apparatus.

It should also be noted that when opaque portion 16 is lifted to a substantially upright position, the surface on which fanciful design 22 (see FIG. 2) is disposed is within the view of patient 26 and thus may serve to distract and/or amuse the patient.

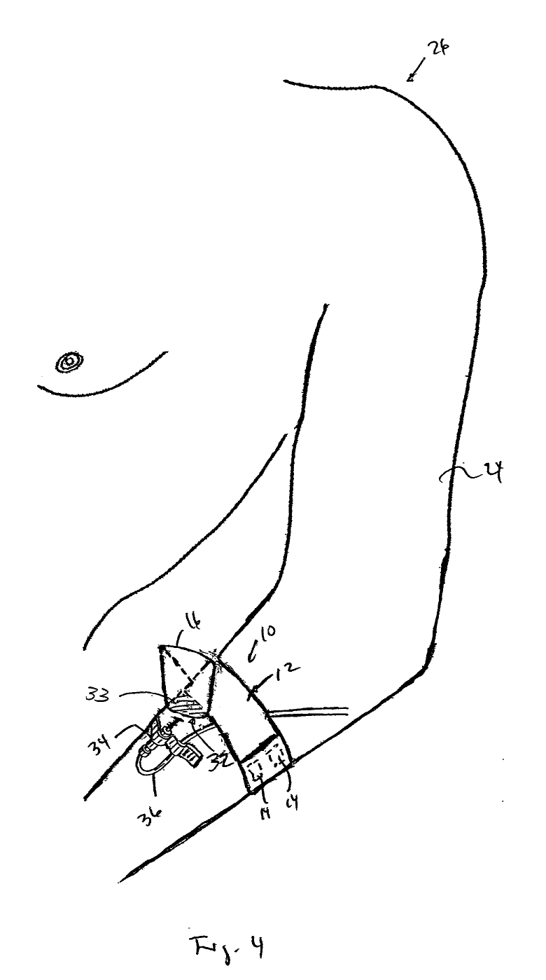

Referring to FIG. 4, device 10 may be used to block the view of patient 26 before catheter 34 penetrates an area of limb 24 of patient 26. According to another aspect of the invention, tube 36 extending from catheter 34 may be secured in place under strap 12 of apparatus 10.

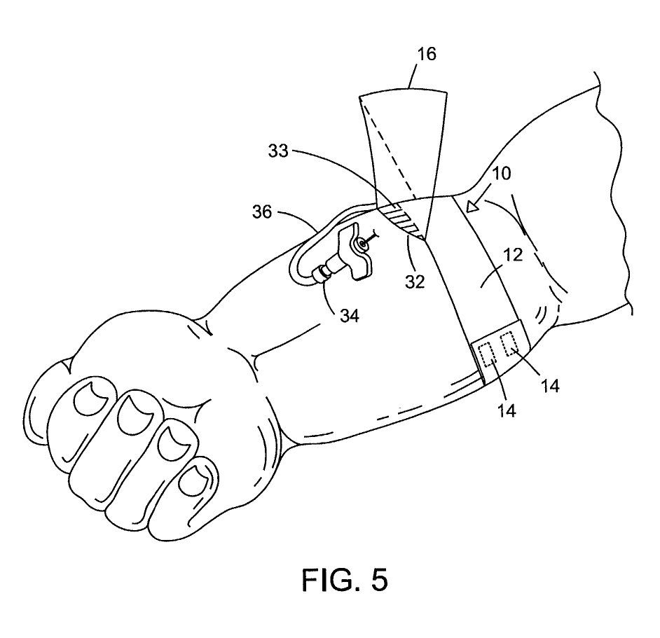

Referring to FIG. 5, device 10 may be used to block the view of insertion site of catheter 34 on a limb of an infant. Similarly, apparatus 10 may be used to block the view of an infant before the needle of a syringe penetrates an area of the infant's body. As can be appreciated by one skilled in the art by providing a device that block the patient's view of the area that is to be penetrated by a needle before the actual penetration, the patient's anxiety may be reduced significantly.

Device 10 is advantageous in that it: blocks a patient's view of the site of insertion of a needle or catheter thereby reducing a patient's anxiety; helps secure intravenous tube with strap 12; can be used with patients of all ages who experience anxiety and discomfort when viewing the site of insertion of a needle or catheter; allows visual and physical access to the insertion site while blocking the patient's view of the same; can be fitted with a cheerful image to amuse and/or distract the patient; can be made in a simple and inexpensive manner; and can be made with disposable material.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

What is claimed is:

1. An apparatus for preventing a patient from viewing a site of penetration of a needle comprising:
means for blocking a patient's view of a preselected region of penetration of a needle; and
means for securing said means for blocking to a limb of said patient;
wherein said means for blocking may be lifted to a position where it may block said patient's view of said preselected region of penetration of a needle after said means for securing has been secured to said patient's limb.

2. The apparatus of claim 1 further comprising means for flexibly connecting said means for blocking to said means for securing to allow said means for blocking to be lifted to said position where it may block said patient's view of said preselected region of penetration.

3. The apparatus of claim 1 wherein said blocking means comprises of a semi-rigid material.

4. The apparatus of claim 1 wherein said blocking means and said securing means are comprised of a semi-rigid cloth.

5. The apparatus of claim 4 wherein said blocking means and said securing means are sewn to one another.

6. The apparatus of claim 1 wherein said blocking means and said securing means are comprised of a disposable material.

7. The apparatus of claim 1 wherein said blocking means includes an image disposed on a surface thereof, said image being visually perceptible by said patient.

8. The apparatus of claim 1 wherein said securing means is a strap having a coupling means for securing said strap to said limb of said patient.

9. The apparatus of claim 8 wherein said coupling means comprises Velcro.

10. The apparatus of claim 8 wherein said coupling means comprises adhesive tape.

11. The apparatus of claim 8 wherein said coupling means comprises snap connectors.

12. An apparatus for preventing a patient from viewing a site of penetration of a needle or catheter comprising:
an opaque member that is capable of blocking a patient's view of a selected region of a limb of said patient that is to be penetrated; and
a strap connected to said opaque member, said strap being securable to said limb of said patient;
wherein said opaque member may be lifted to a position where it may prevent said patient's view of said selected region after said strap has been secured to said limb of said patient.

13. The apparatus of claim 12 wherein a flexible portion is disposed between said strap and said opaque member to allow said opaque member to be lifted to said position where it may prevent said patient's view of said selected region.

14. The apparatus of claim 12 wherein said opaque member comprises a semi-rigid material.

15. The apparatus of claim 12 wherein said opaque member and said strap comprise of cloth.

16. The apparatus of claim 15 wherein said opaque member and said strap are sewn to one another.

17. The apparatus of claim 12 wherein said opaque member and said strap comprise a disposable material.

18. The apparatus of claim 12 wherein said opaque member includes an image disposed on a surface thereof, said image being visually perceptible by said patient.

19. The apparatus of claim 12 wherein said strap includes a coupling device for securing said strap to said limb of said patient.

20. The apparatus of claim 19 wherein said coupling device comprises Velcro.

21. The apparatus of claim 19 wherein said coupling device comprises adhesive tape.

22. The apparatus of claim 19 wherein said coupling device comprises snap connectors.

23. The apparatus of claim 12 wherein said opaque member is trapezium-shaped having a substantially straight side connected to said strap.

24. The apparatus of claim 23 wherein said trapezium-shaped opaque member includes an arcuate edge disposed opposite to said straight side.

25. A method for reducing the anxiety of a patient caused by penetration of a preselected area of said patient's body by a needle or catheter comprising:
securing an opaque member to a patient's limb near said preselected area;
lifting said opaque member to a substantially upright position in order to block said patient's view of said preselected area.

26. The method of claim 25 wherein said opaque member conforms to said patient's limb when it is first secured to said patient's limb, and then forms a substantially upright barrier that blocks said patient's view of said preselected area when it has been lifted to said substantially upright position.

27. The method of claim 25 wherein said opaque member is secured to said patient's limb by a strap.

28. The method of claim 27 further comprising providing a flexible portion between said strap and said opaque member.

29. The method of claim 25 wherein said opaque member is semi-rigid.

30. The method of claim 25 wherein said opaque member is lifted before penetration.
